(19) United States
(12) Patent Application Publication
(10) Pub. No.: US 2010/0234277 A1
(43) Pub. Date: Sep. 16, 2010

(54) COADMINISTRATION OF ALPHA-FETOPROTEIN AND A DISEASE MODIFYING ANTI-RHEUMATIC DRUG FOR TREATING INFLAMMATORY ARTHRITIC DISEASE

(76) Inventors: Mark Moody, Concord, MA (US); Edward J. Stewart, Winchester, MA (US); Brian L. Hamilton, Newton, MA (US); James Murray, Duxbury, MA (US)

Correspondence Address: CLARK & ELBING LLP, 101 FEDERAL STREET, BOSTON, MA, 02110, US

(21) Appl. No.: 12/295,017

(22) PCT Filed: Mar. 27, 2007
(86) PCT No.: PCT/US07/07618

§ 371 Date: Mar. 3, 2009

Related U.S. Application Data
(60) Provisional application No. 60/787,281, filed on Mar. 29, 2006.

(51) Int. Cl. A61K 38/14; A61K 38/16; A61P 29/00; A61P 19/02

(52) U.S. Cl. 514/8; 514/12

(57) ABSTRACT

The present invention relates to methods for treating an anti-inflammatory arthritic disease by coadministering therapeutically effective amounts of an alpha-fetoprotein (or a biologically active fragment thereof) and a disease modifying anti-rheumatic drug (DMARD) to a patient in need thereof. Also disclosed are compositions and kits that include therapeutically effective amounts of an alpha-fetoprotein (or a biologically active fragment thereof) and a DMARD.

### FIELD OF THE INVENTION

This invention relates to treatment methods for treating inflammatory arthritic diseases by coadministering an alpha-fetoprotein, including its biologically active fragments, analogs, and derivatives, with an anti-arthritic drug.

### BACKGROUND OF THE INVENTION

Inflammatory arthritic diseases (e.g., osteoarthritis, rheumatoid arthritis, juvenile rheumatoid arthritis, Crohn's disease, fibromyalgia, systemic lupus erthrematosis, spondylarthropathies (e.g., ankylosing spondylitis), gout, polymyalgia rheumatica, and psoriatic arthritis) are systematic inflammatory conditions that result in swelling, pain, loss of motion, and tenderness of joints and bone tissue throughout the body. Inflammatory arthritic diseases are characterized by hyperplasia and infiltration of the affected tissue with a mixed mononuclear cell infiltrate and destruction of cartilage and bone. In joints, the synovial membrane, which is typically one cell layer thick, develops into a tissue, similar to lymphoid tissue, called the pannus. Pannus is characterized by the presence of newly formed vessels, dendritic cells, T-, B-, and NK-cells, macrophages, and clusters of plasma cells. Beyond the marked changes in synovial tissue and the increased numbers of inflammatory cells there are other immunopathological markers associated with these diseases including antigen-antibody complexes and increased expression of inflammatory cytokines and tissue degrading enzymes. All of these factors likely contribute to the ultimate destruction of the integrity of the joint or bone, resulting in deformity and permanent loss of function. A more detailed description of the etiology and physiology of inflammatory arthritic diseases can be found in Zvaifler, N., “Etiology and Pathogenesis of Rheumatoid Arthritis in Arthritis and Allied Conditions,” 659-73 (ed. D. M. McCarty, Lea and Febiger, Philadelphia, 1989), and Tristano and Fuller, Int. Immunopharmacol. 5:1833-1846, 2006.

Conventional therapeutic strategies for inflammatory arthritic diseases have focused on monotherapies, i.e., administration of a single active compound to treat the disease. The most common monotherapies are based on a class of pharmaceuticals known as DMARDs—disease modifying anti-rheumatic drugs. These pharmaceuticals are generally administered over a period of time, and can, in some cases, provide temporary relief for patients suffering from an inflammatory arthritic disease.

Methotrexate is a common DMARD used for the treatment of an inflammatory arthritic disease. Approximately 70% of individuals with rheumatoid arthritis (RA) respond favorably to treatment with methotrexate and over 50% are still on the drug five years after starting treatment. While the severity of disease does not predict the responsiveness of the patient, more severely affected individuals are typically less responsive to treatment with methotrexate. Furthermore, while methotrexate appears to reduce pain and improve function, there is some controversy about how effective methotrexate is for the prevention of erosive and destructive changes caused by the disease.

A significant patient population is, however, refractory to conventional DMARD monotherapy; they receive only partial or no relief from administration of conventional DMARDs such as methotrexate. Additionally, many patients build up a tolerance to DMARDs, requiring increasingly higher doses. This can create problems for such patients, because stronger doses of DMARDs can lead to increased incidences of undesirable side effects. Thus, there remains a need for new, effective therapeutic approaches for the treatment of inflammatory arthritic diseases. The present invention addresses this and other related needs.

### SUMMARY OF THE INVENTION

In a first aspect, the present invention provides a method of treating, preventing, or reducing one or more symptoms of or the progression of an inflammatory arthritic disease in a patient in need thereof by administering an alpha-fetoprotein (AFP)(or a biologically active fragment thereof) and a DMARD, each in a therapeutically effective amount, to the patient.

Different administration schedules can be followed in the above method. For instance, the AFP or a biologically active fragment thereof or the DMARD can be administered daily, weekly, biweekly, or monthly.

In different embodiments of the above method, the AFP or biologically active fragment thereof, and the DMARD are administered coextensively or separately. Many variations of administration schemes are possible, for example, both the AFP (or biologically active fragment thereof) and the DMARD may be administered initially within the first treatment phase; subsequently, the administration of one (e.g., the AFP or the DMARD) may be terminated while administration of the other is continued. Alternatively, administration of both the AFP (or biologically active fragment thereof) and the DMARD is continued, but one or the other is administered at varying (increased or decreased) dosages.

In a related embodiment of the method, AFP (or a biologically active fragment thereof) and the DMARD are administered in separate dosage forms. In another embodiment of the method, the AFP (or biologically active fragment thereof) and the DMARD are administered in a single dosage form.

In another variation of the method, the AFP (or a biologically active fragment thereof) and the DMARD are administered via two separate routes of administration.

In another example of the method, an AFP (or a biologically active fragment thereof) is administered with one or more DMARDs, each in a therapeutically effective dose, to treat, prevent, or reduce one or more symptoms of or the progression of an inflammatory arthritic disease in a patient in need thereof.

In another aspect, the invention provides a composition that includes an AFP (or a biologically active fragment thereof) and a DMARD, each in an amount that is therapeutically effective to treat, prevent, or reduce the symptoms of or the progression of an inflammatory arthritic disease in a patient in need thereof.

In another embodiment, the composition includes an AFP (or a biologically active fragment thereof) and one or more DMARDs, each in an amount that is therapeutically effective to treat, prevent, or reduce the symptoms of or the progression of an inflammatory arthritic disease in a patient in need thereof.

Another aspect of the invention features a kit that includes 1) an AFP or a biologically active fragment thereof and a DMARD, each in a therapeutically effective amount to treat, prevent, or reduce the symptoms of or the progression of an inflammatory arthritic disease in a patient in need thereof, and 2) instructions for administration of the AFP or a biologically active fragment thereof and the DMARD to the patient.

In other examples of the kit, the AFP (or a biologically active fragment thereof) and the DMARD are formulated in a single dosage form or in separate dosage forms.

In other examples, the kit of the present invention comprises an AFP (or a biologically active fragment thereof) and one or more DMARDs, each in a therapeutically effective amount to treat, prevent, or reduce the symptoms of or the progression of an inflammatory arthritic disease in a patient, and instructions for the use of the kit.

In all the above embodiments of the invention, the AFP (or a biologically active fragment thereof) may be a human recombinant AFP having an amino acid sequence substantially identical (e.g., 60% identical, preferably 65%, 70%, 75%, 80%, 85%, 90%, 91%, 92%, 93%, 94%, 95%, 96%, 97%, 98%, 99%, or even 100% identical) to SEQ ID NO: 1 or the AFP may be isolated from a naturally-occurring source. In other embodiments, the AFP (or biologically active fragment thereof) is non-glycosylated.

In addition, in all the embodiments described above, the DMARD may be auranofin, aurothioglucose, azathioprine, chlorambucil, cyclophosphamide, cyclosporine, D-penicillamine, gold sodium thiomalate (injectable gold), hydroxychloroquine, leflunomide, methotrexate, minocycline, mycophenolate mofetil, or sulfasalazine.

In all aspects of the invention, an inflammatory arthritic disease is osteoarthritis, rheumatoid arthritis (RA), juvenile rheumatoid arthritis, Crohn's disease, fibromyalgia, systemic lupus erthrematosis, spondylarthropathies (e.g., ankylosing spondylitis), gout, polymyalgia rheumatica, or psoriatic arthritis.

A variety of different routes of administration and formulations may be useful for the above methods, compositions, and kits of the present invention. For instance, the AFP (or biologically active fragment thereof) or the DMARD may be administered to a patient intravenously, intramuscularly, orally, by inhalation, parenterally, intraperitoneally, intraarterially, transdermally, sublingually, nasally, through use of suppositories, transbuccally, liposomally, adiposally, intaocularly, subcutaneously, intrathecally, topically, or through local administration; and compositions and kits containing the AFP (or biologically active fragment thereof) or the DMARD may be formulated for intravenous, intramuscular, oral, parenteral, intraperitoneal, intraarterial, transdermal, sublingual, nasal, transbuccal, liposomal, adiposal, intraocular, subcutaneous, intrathecal, topical, or through suppository, inhalation, or local administration. In a further embodiment of all aspects of the invention, the patient is diagnosed with an inflammatory arthritic disease prior to treatment.

### DEFINITIONS

The term “disease modifying anti-rheumatic drug” or “DMARD” refers to a therapeutic agent that is used for the treatment of an inflammatory disease. A DMARD can be used treat, prevent, or reduce one or more of the symptoms of or the progression of an inflammatory arthritic disease in a patient when administered in a therapeutically effective amount. Examples of DMARDs known in the art include auranofin, aurothioglucose, azathioprine, chlorambucil, cyclophosphamide, cyclosporine, D-penicillamine, gold sodium thiomalate (injectable gold), hydroxychloroquine, leflunomide, methotrexate, minocycline, mycophenolate mofetil, or sulfasalazine.

By “administered coextensively” is meant the administration of two or more therapeutic agents across time periods that completely overlap or at least in part overlap. When the therapeutic agents are “administered separately,” the two or more therapeutic agents are administered in time periods that do not overlap. In certain embodiments of separate administration, the therapeutic agents are administered in time periods that do not overlap, but are within the bioactive period for each respective agent, e.g., the latter administered agent is administered before the plasma concentration of the earlier administered agent(s) decreases to less than about 60%, more preferably to less than about 50%, 40%, 30%, and most preferably to less than about 20% or 10%. In other cases of separate administration, the agents are administered outside of their respective bioactive periods.

The term “inflammatory arthritic disease” refers to a chronic and systemic disease in which a person's immune system attacks its own tissues (e.g., bone tissue or soft tissues of the joint). Inflammatory arthritic diseases are characterized by inflammation of the lining, or synovium, of the joints or bone tissue, which can lead to long-term joint or bone damage, resulting in chronic pain, loss of function, and disability. Examples of inflammatory arthritic disease include osteroarthritis, rheumatoid arthritis, juvenile rheumatoid arthritis, Crohn's disease, fibromyalgia, systemic lupus erythematosus, spondylarthropathies ankylosing spondylitis), gout, polymyalagia rheumatica, and psoriatic arthritis.

The term “alpha-fetoprotein” or “AFP,” as used in this application, refers to a polypeptide having an amino acid sequence substantially identical to the mature human AFP (SEQ ID NO: 1), which was first described by Pucci et al. (Biochemistry 30:5061-5066, 1991), or a nucleic acid that encodes the polypeptide (NCBI Accession No. NM_{—}001134; SEQ ID NO: 2) (see, FIG. 1). An AFP polypeptide of this invention has an amino acid sequence that is substantially identical to SEQ ID NO: 1. The AFP term is not limited to the full length sequence; it also includes any recombinant or naturally occurring human AFP (whether or not having the same post-translational modifications as the naturally occurring version) and biologically active variants of human AFP (e.g., a non-glycosylated form of AFP, as well as fragments, analogs, and derivatives).

In some embodiments, the AFP of this invention may contain modifications of the amino acid sequence of SEQ ID NO: 1, including substitutions (e.g., conservative substitutions), deletions, or additions of one or more amino acid residues. For instance, a recombinant human AFP is described in U.S. Patent Application Publication No. 2004/0098755, incorporated by reference, which contains an asparagine to glutamine substitution at position 233 of SEQ ID NO: 1. The term “alpha-fetoprotein” also encompasses any derivatives or analogues of AFP described herein. Also, encompassed by the present invention is the use of a non-glycosylated AFP in which the asparagine at position 233 of SEQ ID NO: 1 is substituted with a residue other than asparagine (SEQ ID NO: 12). For example, a non-glycosylated AFP has an asparagine to glutamine substitution at residue 233 of SEQ ID NO: 1.

An AFP polypeptide of this invention has the same or similar biological activity as the native human AFP in the ability to bind to human leukocytes and in the ability to suppress autoimmune reactions. The leukocyte binding assays used for testing AFP activity is described in, e.g., Parker et al., Protein Express. Purification 38:177-183, 2004; and is described in detail in this application. The desired autoimmune suppression activity for an AFP polypeptide of this application is demonstrated either by the polypeptide's ability to suppress human autologous mixed lymphocyte reactions (AMLR) or by the polypeptide's ability to suppress experimental autoimmune encephalomyelitis (EAE) in a mouse model. Such activity can be verified by assays described herein. A functional AFP polypeptide within the meaning of this application demonstrates at least 40%, 50%, 60%, 70%, 80%, 90%, or 100% of the ability of the native human AFP to bind human monocytes in an assay described in Parker et al., supra, and at least 40%, 50%, 60%, 70%, 80%, 90%, or 100% of the ability of the native human AFP to suppress autoimmune reactions. The latter activity is shown by suppression of human AMLR in an assay described in U.S. Pat. No. 5,965,528, or, in the alternative, by suppression of the development of EAE in a mouse model (see, e.g., Fritz et al., J. Immunol. 130:1024, 1983; Naiki et al., Int. J. Immunopharmacol. 13:235, 1991; and Goverman, Lab. Anim. Sci., 46:482, 1996).

By the term “amino acid” is meant any naturally occurring or synthetic amino acid, as well as any amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure different from the general chemical structure of an amino acid, but are capable of functioning in a manner similar to a naturally occurring amino acid. An AFP or a biologically active fragment thereof of the invention can include naturally occurring or synthetic amino acids or amino acid mimetics.

By the term “biologically active fragment thereof” is meant a fragment of an AFP which is biologically active. Biological activity of the AFP fragment can be determined through the assays described above for an AFP (e.g., AMLR assays, AFP binding to monocyte assays, and experiments using the EAE mouse model) and as described herein. A typical biologically active AFP fragment contains at least 5 contiguous amino acids of SEQ ID NO: 1, or at least 8 contiguous amino acids, preferably at least 10, 20, or 50 contiguous amino acids, more preferably at least 100 contiguous amino acids, and most preferably at least 200, 300, 400, or more contiguous amino acids in length. For instance, U.S. Pat. No. 6,818,741 discloses an 8-amino acid fragment of human AFP (amino acids 471-478; EMTPVNPG; SEQ ID NO: 3), as well as other AFP fragments containing this 8-mer. An active AFP fragment of this invention may further contain amino acid substitution, deletion, or addition at a limited number of positions, so long as the AFP fragment has at least 90% identity to its corresponding portion within SEQ ID NO: 1. For sequence comparison purposes in this application, the corresponding sequence of SEQ ID NO: 1 is deemed to have the same number of amino acids as a given AFP fragment. For instance, a 34-mer AFP peptide corresponding to the 446-479 segment of SEQ ID NO: 1 (LSEDKLLACGEGAADIIIGHLCIRHEMTPVNPGV; SEQ ID NO: 4) may contain up to 3 amino acids altered from the 446-479 segment of SEQ ID NO: 1. One such example of sequence deviation in biologically active AFP fragments is found in U.S. Pat. No. 5,707,963, which discloses a 34-amino acid fragment of human AFP (SEQ ID NO: 4) with flexibility at two amino acid residues (9 and 22). Some other examples of AFP fragments include Domain I (amino acids 2-198 of mature human AFP; SEQ ID NO: 5), Domain II (amino acids 199-390 of mature human AFP; SEQ ID NO: 6), Domain III (amino acids 391-591 of mature human AFP; SEQ ID NO: 7), Domain I+II (amino acids 2-390 of mature human AFP; SEQ ID NO: 8), Domain II+III (amino acids 199-591 of mature human AFP; SEQ ID NO: 9), and human AFP Fragment I (amino acids 267-591 of mature human AFP; SEQ ID NO: 10) (see, FIG. 2).

By “substantial identity” or “substantially identical,” is meant that the polynucleotide or polypeptide sequence has the same polypeptide or polynucleotide sequence, respectively, as the reference sequence or has a specified percentage of nucleotides or amino acid residues, respectively, that are the same at the corresponding locations within the reference sequence when the two sequences are optimally aligned. For instance, an amino acid sequence that is “substantially identical” to a reference sequence has at least about 60% identity, preferably 65%, 70%, 75%, 80%, 85%, 90%, 91%, 92%, 93%, 94%, 95%, 96%, 97%, 98%, 99%, or higher percentage identity (up to 100%) to a reference sequence (e.g., the mature human AFP amino acid sequence as set forth in SEQ ID NO: 1, the human AFP nucleic acid sequence set forth in SEQ ID NO: 2, or a pre-determined segment of SEQ ID NOS: 1 or 2), when compared and aligned for maximum correspondence over the full length of the reference sequence as measured using BLAST or BLAST 2.0 sequence comparison algorithms with default parameters, or by manual alignment and visual inspection (see, e.g., NCBI; http://www.ncbi.nlm.nih.gov/blast/bl2seq/wblast2.cgi).

The term “methotrexate” refers to the pharmaceutical N-[4-[[(2,4-diamino-6-pteridinyl)methyl]methylamino]benzoy]-L-glutamic acid. In the context of the invention, methotrexate also refers to pharmaceuticals that are analogs, derivatives, and prodrugs of N-[4-[[(2,4-diamino-6-pteridinyl)methyl]methylamino]benzoyl]-L-glutamic acid that may be used in the practice of this invention. For example, prodrugs of N-[4-[[(2,4-diamino-6-pteridinyl)methyl]methylamino]benzoyl]-L-glutamic acid may be used to increase bioavailability through selective bioconversion.

A “therapeutically effective amount” of a therapeutic agent is an amount of the agent that is sufficient to effectuate a desired therapeutic effect on a given condition or disease. Such amount may vary depending on the effect to be achieved. For instance, a “therapeutically effective amount” of a DMARD for treating an inflammatory arthritic disease alone may be different from the “therapeutically effective amount” of a DMARD for treating the same condition when used in combination with an AFP (or a biologically active fragment thereof). In different embodiments, the therapeutic effect is to treat, prevent, or reduce the symptoms (e.g., pain and inflammation in the affected tissues) or progression of an inflammatory arthritic disease in a patient.

### BRIEF DESCRIPTION OF THE FIGURES

FIG. 1 shows the amino acid sequence (SEQ ID NO: 1) and the mRNA nucleic acid sequence of mature human AFP (SEQ ID NO: 2).

FIG. 2 shows the amino acid sequences of biologically active fragments of AFP including the amino acids 2-198 (Domain I; SEQ ID NO: 5), amino acids 199-390 (Domain II; SEQ ID NO: 6), amino acids 391-591 (Domain III; SEQ ID NO: 7), amino acids 2-390 (Domains I+II; SEQ ID NO: 8), amino acids 199-591 (Domains II+III; SEQ ID NO: 9), and amino acids 261-591 of mature human AFP (Human AFP Fragment 1; SEQ ID NO: 10).

FIG. 3 shows the difference between the observed versus the expected inhibition of phytohemagluttinin-L-induced TNF-a production following treatment of splenocytes with different dosage combinations of a biologically active AFP fragment (final concentrations from 1 to 100 μg/mL) and methotrexate (MTX; final concentrations from 1 to 20 μM), when the Highest Single Agent definition was used for comparison of the data. Difference values less than or equal to 5 percentage points are considered additive, values greater than 5 percentage points are considered synergistic, and values less than −5 percentage points are considered antagonistic. Specific dosage combinations of the biologically active AFP fragment and MTX are indicated as being additive, synergistic, or antagonistic.

FIG. 4 shows the difference between the observed versus the expected inhibition of phytohemagluttinin-L-induced TNF-α production following treatment of splenocytes with different dosage combinations of a biologically active AFP fragment (final concentrations from 1 to 100 μg/mL) and methotrexate (MTX; final concentrations from 1 to 20 μM), when the Bliss Independence definition was used for comparison of the data. Difference values less than or equal to 5 percentage points are considered additive, values greater than 5 percentage points are considered synergistic, and values less than −5 percentage points are considered antagonistic. Specific dosage combinations of the biologically active AFP fragment and MTX are indicated as being additive, synergistic, or antagonistic.

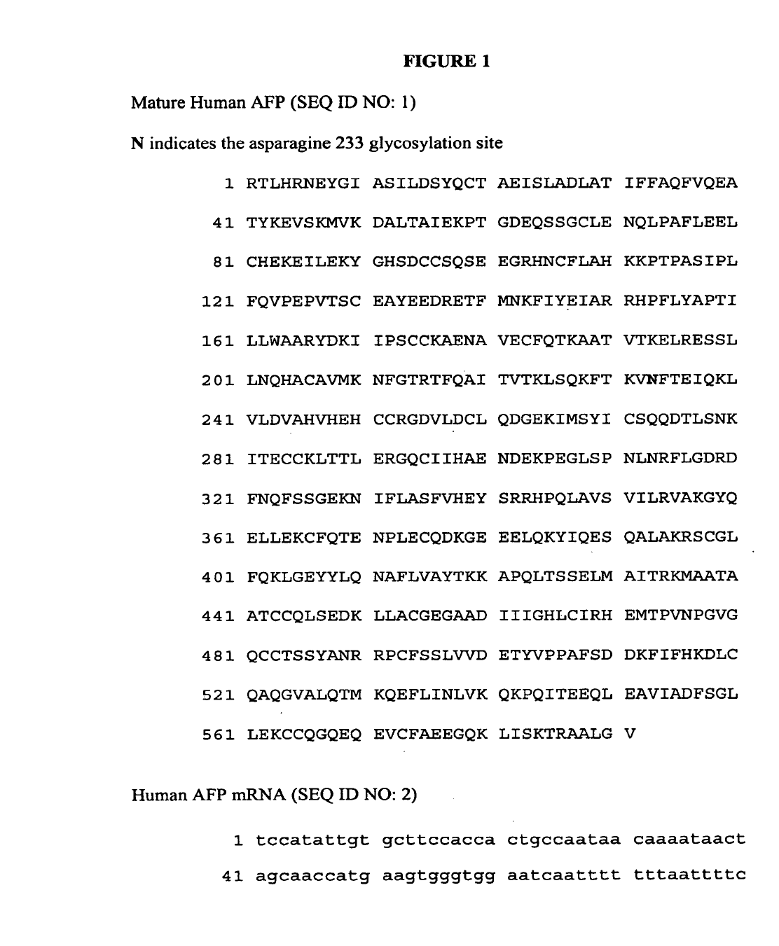

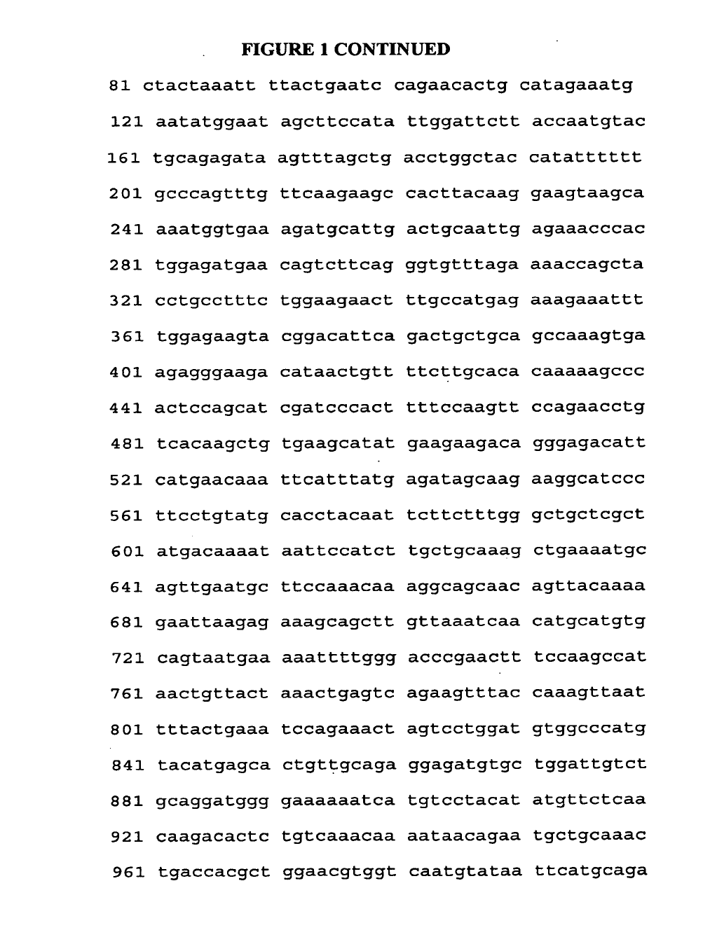

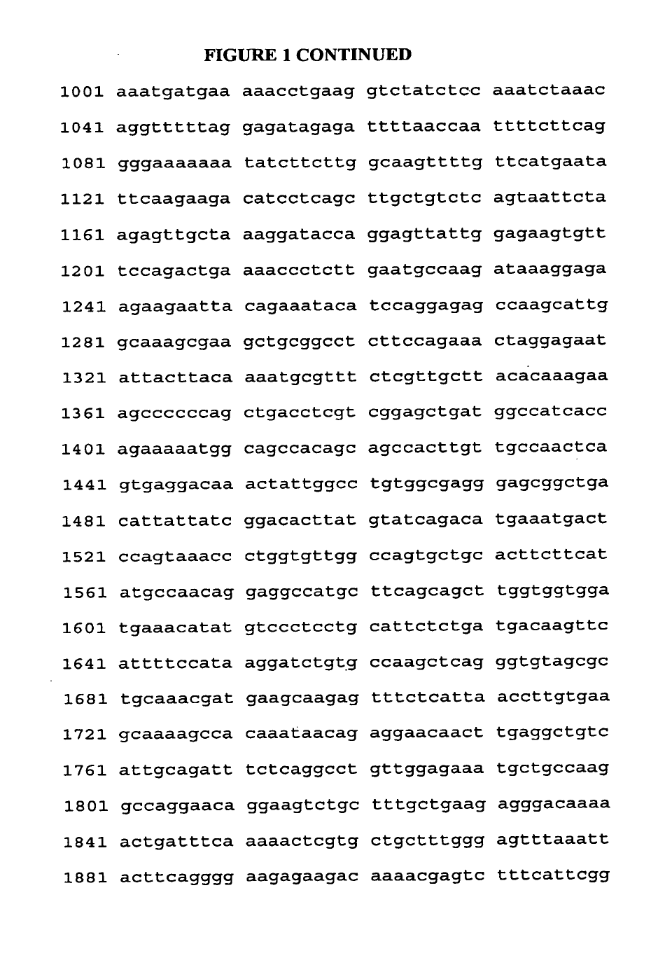

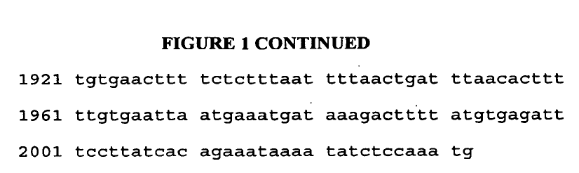

### DETAILED DESCRIPTION

The inventors have discovered that AFP and DMARD combination therapy can be an effective treatment for inflammatory arthritic diseases (e.g., RA). When co-administered, AFP and DMARDs act synergistically to treat inflammatory arthritic diseases by, e.g., allowing the administration of lower dosages of one or both therapeutic agents to the patient, relative to the monotherapy dosage of AFP or the DMARD administered to treat inflammatory arthritic disease in a patient, or by reducing the side effects associated with monotherapy using AFP or the DMARD, even when the AFP and DMARD are administered at their normal monotherapy dosages.

Accordingly, the invention provides a combination treatment method for inflammatory arthritic diseases which differs from the previously known therapeutic strategies of administering a DMARD or an AFP alone to treat these conditions. This combination treatment method involves co-administering one or more DMARDs and an AFP (or a biologically active fragment thereof), each in a therapeutically effective amount, to a patient in need thereof.

In another aspect, the invention provides a pharmaceutical composition that includes both a DMARD and an AFP (or a biologically active fragment thereof), each in a therapeutically effective amount for treating, preventing, or reducing the symptoms of or the progression of an inflammatory arthritic disease. Such a composition optionally contains one or more pharmaceutically acceptable excipients and is formulated to be administered intravenously, intramuscularly, orally, by inhalation, parenterally, intraperitoneally, intraarterially, transdermally, sublingually, nasally, through the use of suppositories, transbucally, liposomally, adiposally, intraocularly, subcutaneously, intrathecally, topically, or through local administration.

In a further aspect, the invention provides a kit for treating an inflammatory arthritic disease, which includes a therapeutically effective amount of a DMARD and an AFP (or a biologically active fragment thereof), along with proper instructions for the use of the kit.

In all of the above aspects, the DMARD is auranofin, aurothioglucose, azathioprine, chlorambucil, cyclophosphamide, cyclosporine, D-penicillamine, gold sodium thiomalate (injectable gold), hydroxychloroquine, leflunomide, methotrexate, minocycline, mycophenolate mofetil, or sulfasalazine.

In all of the above aspects, one or more DMARDs may be provided with an AFP (or a biologically active fragment thereof), each in a therapeutically effective amount.

In each embodiment, the AFP (or biologically active fragment thereof) and the DMARD may be provided in a single dosage form or in separate dosage forms (coextensively and non-coextensively) and the AFP (or a biologically active fragment thereof) and the DMARD may be administered one or more times daily, weekly, biweekly, or monthly. The concentration of DMARD or AFP administered during a treatment period can be varied (e.g., both increased, both decreased, or one increased and the other decreased). In an embodiment, the concentration of the DMARD, e.g., methotrexate, is decreased relative to its monotherapy dosage in the combination therapy with AFP.

### Diagnosis and Monitoring of Inflammatory Arthritic Diseases

Inflammatory arthritic diseases are a class of diseases that affect the joints or bone tissue of a patient with a high prevalence in the general population, particularly among the older age groups. These diseases progress through stages: an early stage is the inflammation of the synovial lining or bone tissue, causing pain, warmth, stiffness, redness, and swelling around the affected tissue. In a later phases, the inflamed cells release enzymes that may digest bone and cartilage, often causing the involved joint or bone to lose its shape and alignment, which frequently leads to more pain and loss of movement. Because the general methodology for diagnosing the various inflammatory arthritic diseases is well established and routinely practiced by clinicians, it is only briefly described below.

There is no single test for diagnosing inflammatory arthritic diseases. A proper diagnosis is made based on clinical symptoms, which can be revealed through a physical examination and a review of the patient's medical history, as well as through various laboratory test results.

The symptoms of inflammatory arthritic diseases can vary but generally include swelling, tenderness, and loss of motion. The symptoms may start in any joint or bone, but most commonly begins in the smaller joints and bones of the fingers, hands, and wrists. Such joint and bone involvement is usually symmetrical. Other common physical symptoms of include: fatigue; stiffness, particularly in the morning and when sitting for long periods of time; weakness; flu-like symptoms, including a low-grade fever; pain associated with prolonged sitting; the occurrence of flares of disease activity followed by remission or disease inactivity; rheumatoid nodules, or lumps of tissue under the skin; muscle pain; loss of appetite, depression, weight loss, anemia, cold and/or sweaty hands and feet; and involvement of the glands around the eyes and mouth, causing decreased production of tears and saliva. Advanced changes associated with these diseases include damage to bone and to cartilage, tendons, and ligaments, which causes deformity and instability in the joints, leading to limited range of motion.

Another important tool in diagnosing inflammatory arthritic diseases is laboratory testing. The most commonly used tests include the following:

Complete Blood Count: Patients with an inflammatory arthritic disease often have a low red blood count, whereas white blood cell count may be high, signaling that infection is present in the body. Platelet count may also be elevated due to the inflammation.

Erythrocyte Sedimentation Rate (ESR): ESR measures the speed at which red blood cells fall to the bottom of a test tube. An elevated ESR is observed in about 60% of patients with rheumatoid arthritis (i.e., an inflammatory arthritic disease). A high ESR indicates inflammation and increased severity of the disease. ESR readings can also be used to monitor the progress and effectiveness of a treatment regiment.

C-Reactive Protein (CRP): CRP is found in the body and becomes elevated when inflammation is present. The level of CRP positively correlates with the severity of the disease. Although ESR and CRP reflect similar degrees of inflammation, sometimes a patient with an inflammatory arthritic disease may have an elevated reading in one test but not the other. The CRP test may be repeated regularly to monitor inflammation and a patient's response to medication.

Rheumatoid Factor (RF): Approximately 70 to 80% of people with RF present in their body have rheumatoid arthritis (i.e., an inflammatory arthritic disease). The higher levels of RF present in a patient suffering from an inflammatory arthritic disease indicate heightened severity of disease. Patients that do not have a detectable amount of RF in their blood and are referred to as “seronegative;” otherwise, they are referred to as “seropositive.”

Antinuclear Antibodies (ANA): This test detects a group of autoantibodies, which is seen in about 30 to 40% of people with rheumatoid arthritis (i.e., an inflammatory arthritic disease). Although it is commonly used as a screening tool, ANA testing is not used as a diagnostic tool because many people without an inflammatory arthritic disease or with other diseases can have ANAs.

Imaging Studies: Radiographs (X-rays), magnetic resonance imaging (MRI), joint ultrasound, bone densitometry (DEXA), etc., may be used to measure the amount of joint or bone tissue damage in a patient with an inflammatory arthritic disease.

All of the methodologies described above are also useful for monitoring the progression of the conditions of a patient with an inflammatory arthritic disease, such that the effectiveness of the treatment they are receiving can be assessed.

### AFP Compositions

Mature human AFP is a protein of 591 amino acids (see, SEQ ID NO: 1), resulting from a precursor of 609 amino acids (GenBank Accession No. NP_{—}001125; SEQ ID NO: 11) having an 18-amino acid signal sequence cleaved off. The full length polynucleotide sequence encoding for this protein was first identified by Morinaga et al. (Proc. Natl. Acad. Sci., USA 80:4604-4608, 1983). The amino acid sequence for the mature human AFP is provided by, e.g., Pucci et al., Biochemistry 30:5061-5066, 1991; and Parker et al., Protein Express. Purification 38:177-183, 2004.

For the purpose of practicing the present invention, both naturally occurring human AFP and recombinantly produced AFP polypeptides or biologically active fragments thereof can be used. The naturally occurring human AFP can be obtained through purification from, e.g., umbilical cords or umbilical cord serum; whereas a recombinant AFP polypeptide or fragment can be obtained through a prokaryotic or eukaryotic expression system, such as those described in, e.g., U.S. Pat. No. 5,384,250 and U.S. Patent Application Publication No. 20040098755, which include methods for purifying AFP from the biological fluids of transgenic mammals, and other methods known in the art. Use of different expression systems may result in different post-translational modification of the recombinant protein or fragment. For instance, naturally occurring human AFP is a variably glycosylated protein. In contrast, the recombinant AFP or fragment may be unglycosylated when produced by a prokaryotic host cell or may be somewhat differently glycosylated when produced by a eukaryotic host cell. Alternatively, a recombinant AFP can be genetically modified to eliminate glycosylation (e.g., by eliminating the single glycosylation site), regardless of the expression system in which it is produced. Human AFP is available through various commercial suppliers, including Fitzgerald Industries International (Concord, Mass.), Cell Sciences (Canton, Mass.), and Biodesign International (Saco, Me.), and can be purified from the milk of transgenic animals expressing recombinant human AFP (Merrimack Pharmaceuticals, Cambridge, Mass.).

Furthermore, it is possible to employ well-known chemical synthesis methods to synthesize an AFP polypeptide or fragment, particularly when the AFP fragment is a peptide of a relatively short length, e.g., having less than 100 or 50 amino acids.

Any AFP polypeptide or fragment thereof; regardless of its origin or status of post-translational modification, can be used in the present invention if the polypeptide has the same or comparable activity to a substantial degree (e.g., at least 40%, desirably at least 50%, 60%, 70%, and more desirably at least 80%, 90%, 95%, or 100% or more) as the native human AFP in binding to human leukocytes and in suppressing human autologous mixed lymphocyte reactions (AMLR) or suppressing EAE in a mouse model. The cell binding assay and autoimmune suppression assays are discussed in more detail below.

Similarly, fragments of the human AFP can also be used in the treatment method of the present invention, so long as the fragments retain a substantial portion of the biological activity (e.g., at least 60%, preferably 70%, 80%, or 90%, and more preferably 95% or 100% or more of the biological activity) of the naturally occurring human AFP in the human leukocyte binding assay and in the AMLR or mouse EAE assays. Fragments of human AFP can be generated by methods known to those skilled in the art, e.g., proteolytic cleavage or recombinant expression, or may result from normal protein processing (e.g., removal from a nascent polypeptide or amino acids that are not required for biological activity). Fragments of human AFP can also be produced recombinantly using the techniques described above. Chemical methods can also be useful for synthesizing active AFP fragments.

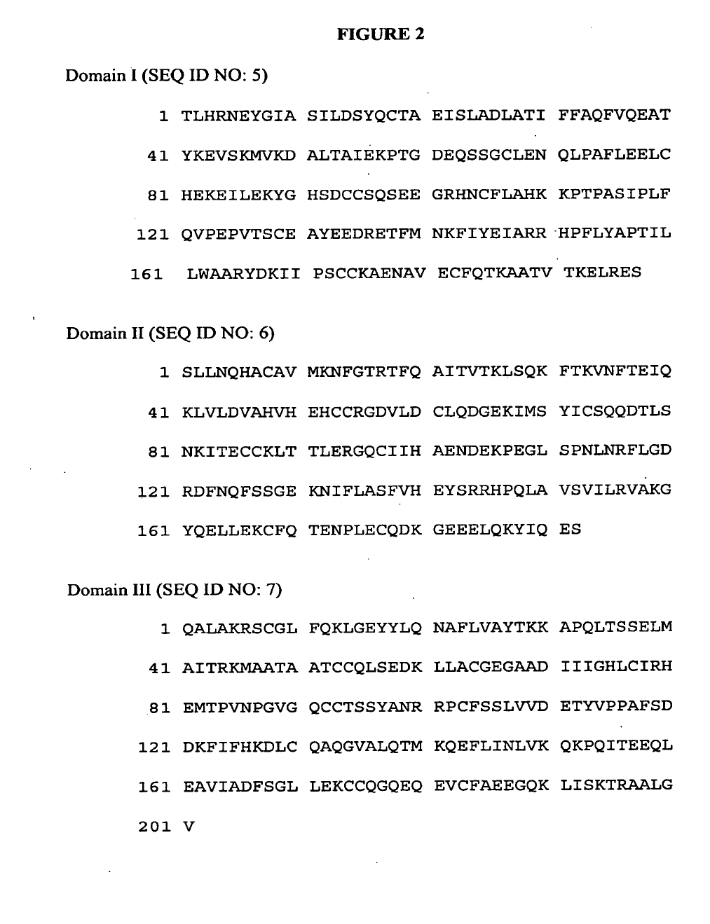

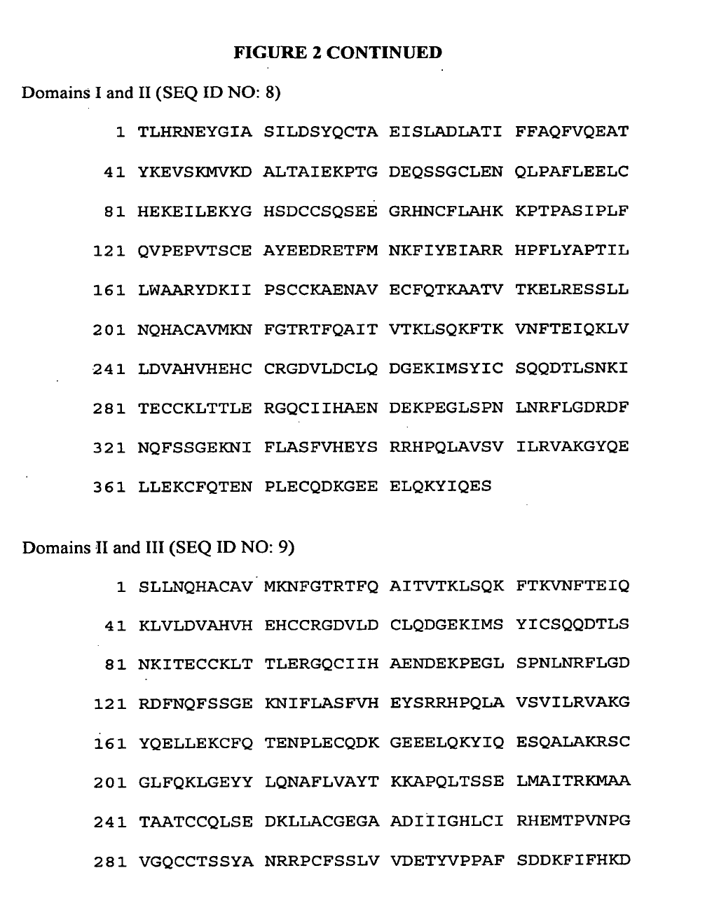

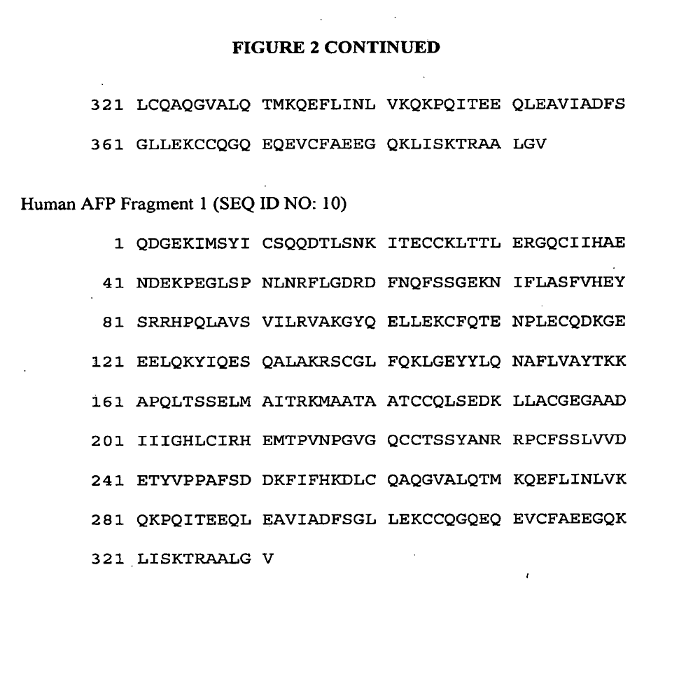

Examples of human AFP fragments suitable for use in practicing the present invention are shown in FIG. 2 and include Domain I (amino acids 2-198 of mature human AFP; SEQ ID NO: 5), Domain II (amino acids 199-390 of mature human AFP; SEQ ID NO: 6), Domain III (amino acids 391-591 of mature human AFP; SEQ ID NO: 7), Domain I+II (amino acids 2-390 of mature human AFP; SEQ ID NO: 8), Domain II+III (amino acids 199-591 of mature human AFP; SEQ ID NO: 9), and Human Recombinant AFP Fragment I (amino acids 267-591 of mature human AFP; SEQ ID NO: 10). Other examples of known AFP fragments can be found in, e.g., U.S. Pat. No. 5,707,963 and U.S. Pat. No. 6,818,741, herein incorporated by reference.

Also encompassed with the claimed invention is the use of functional derivatives or analogs of full length human AFP or fragments thereof. As described in earlier sections, such derivatives or analogs can differ from the full-length native human AFP or portions thereof by amino acid sequence differences (e.g., additions, deletions, conservative or non-conservative substitutions), or by modifications (e.g., post-translational modifications) that do not affect sequence, or by both. The derivatives/analogs of the invention will generally exhibit at least 90%, more preferably at least 95%, or even 99% amino acid identity with all or part of the native human AFP amino acid sequence (SEQ ID NO: 1). Some preferred functional AFP derivatives contain one or more conservative substitutions, in which certain amino acid residues are substituted by other residues having similar chemical structures (e.g., alanine for glycine, arginine for lysine, etc.). The derivatives/analogs mentioned above may include allelic variants, inter-species variants, and genetic variants, both natural and induced (for example, resulting from random mutagenesis by, e.g., site-specific mutagenesis according to methods described in scientific literature, such as Sambrook et al., Molecular Cloning, A Laboratory Manual, 3rd ed., 2001; Kriegler, Gene Transfer and Expression: A Laboratory Manual, 1990; and Ausubel et al., eds., Current Protocols in Molecular Biology, 1994.

In addition, the AFP and AFP fragments described herein, may also contain additional heterologous sequences which provide additional activity or function to the AFP or AFP fragment. For example, the heterologous sequence may be a detectable protein (e.g., green fluorescence protein, hemagluttinin, or alkaline phosphatase) or may stabilize the polypeptide or facilitate its production or purification (e.g., His₆, a myc tag, streptavidin, or a secretion signal). Heterologous sequences may also be added to enhance solubility or increase half-life, for example, hydrophilic amino acid residues (see, e.g., Murby et al., Eur. J. Biochem. 230:38-44, 1995), glycosylation sequences (see, Sinclair and Elliott, J. Pharm. Sci. 94:1626-1635, 2005), or the carboxy terminus of human chorionic gonadotropin or thrombopoeitin (see, Lee et al., Biochem. Biophys. Res. Comm. 339:380-385, 2006). These AFP or AFP fragments containing heterologous sequences may be produced by any standard method in the art.

For production of stable cell lines expressing the AFPs or AFP fragments (with or without additional heterologous sequences) described above, PCR-amplified nucleic acids encoding any of the AFPs or AFP fragments described above may be cloned into the restriction site of a derivative of a mammalian expression vector. For example, KA, which is a derivative of pcDNA3 (Invitrogen, Carlsbad, Calif.) contains a DNA fragment encoding an influenza virus hemagluttinin (HA). Alternatively, vector derivatives encoding other tags, such as c-myc or poly-histidine tags, can be used.

An AFP, an AFP fragment, or an AFP derivative/analog may differ from a naturally occurring human AFP due to post-translational modifications (which do not normally alter primary sequence), which include in vivo, or in vitro chemical derivatization of polypeptides, e.g., acetylation, carboxylation, pegylation; such modifications may occur during polypeptide synthesis or processing or following treatment with isolated modifying enzymes. Also included are cyclized peptide molecules and analogs that contain residues other than L-amino acids, e.g., D-amino acids or non-naturally occurring or synthetic amino acids, e.g., β or γ amino acids, or L-amino acids with non-natural side chains (see e.g., Noren et al., Science 244:182, 1989). Methods for site-specific incorporation of non-natural amino acids into the protein backbone of proteins is described, e.g., in Ellman et al., Science 255:197, 1992. Also included are chemically synthesized polypeptides or peptides with modified peptide bonds (e.g., non-peptide bonds as described in U.S. Pat. No. 4,897,445 and U.S. Pat. No. 5,059,653) or modified side chains to obtain the desired pharmaceutical properties as described herein. Useful AFPs, AFP fragments, or AFP derivatives and analogs are identified using art-recognized methods, e.g., those described below, to confirm the desired functionality for binding to human leukocytes and for suppressing autoimmune reaction in either human autologous mixed lymphocyte reactions (AMLR) or an experimental autoimmune encephalomyelitis (EAE) in a mouse model.

### AFP Activity Assays

As stated above, AFP polypeptides or AFP fragments suitable for use in the method of the present invention may include various derivatives, analogs, or fragments of the naturally occurring human AFP, so long as the polypeptides or fragments retain a biological activity of mature human AFP. AFP protein or AFP fragment biological activity is demonstrated by their ability to bind human leukocytes, to inhibit human autologous mixed lymphocyte reactions (AMLR), or to inhibit experimental autoimmune encephalomyelitis (EAE) in a mouse model.

A first assay of AFP polypeptide or fragment activity is the measurement of its ability to specifically bind to cellular receptors on human peripheral monocytes. A binding assay suitable for this purpose is described in Parker et al., Protein Express. Purification 38:177-183, 2004. Briefly, a competitive assay format is used to test a candidate AFP polypeptide for its ability to specifically bind to U937 cells, a human monocytic cell line. The cells are maintained in RPMI media with 10% fetal bovine serum. Prior to the binding assay, the cells are washed twice with serum-free media and adjusted to 2.5×10⁶ cells/ml in phosphate-buffered saline (PBS). Native human AFP (SEQ ID NO: 1) or non-glycosylated human AFP (see, e.g., SEQ ID NO: 12, where, e.g., residue 233 is glutamine) is labeled with a detectable label, e.g., fluorescein, in a proper reaction followed by removal of the unattached labeling material, for instance, by gel filtration. In the case of labeling human AFP with fluorescein, the protein is mixed with a solution of fluorescein-5-isothiocyanate in dimethyl sulfoxide for 1 hour in the dark, followed by gel filtration to remove unbound dye. Labeled human AFP is stored in 20% glycerol at −20° C. until use. For the binding assay, a certain number of U937 cells (e.g., 40 μl of cell suspension at 2.5×10⁶ cells/ml concentration) are mixed with a pre-determined amount of labeled human AFP (e.g., at a final concentration of 0.5 μM) with unlabeled human AFP or unlabeled candidate AFP polypeptide or fragment, each at a set of final concentrations (e.g., 20, 10, 5, 2.5, 1.25, and 0.625 μM) to determine the IC₅₀ values for both human AFP and the candidate AFP polypeptide or fragment. At the conclusion of the binding process, the cells are then washed with PBS and suspended in fresh PBS so that the labeled AFP remaining on U937 cells can be measured, e.g., by flow cytometry.

A second assay of AFP polypeptide or fragment activity is the measurement of its ability to suppress autoimmune reactions, either in AMLR or in a mouse model of EAE. Methods are known in the art for testing AMLR and its inhibition. For instance, U.S. Pat. Nos. 5,965,528 and 6,288,034 describe the AMLR system as follows: isolation of human peripheral blood mononuclear cells (PBMC), their fractionation into non-T cell populations, and the AMLR, performed according to standard procedures. Briefly, responder T cells are isolated by passing 1.5×10⁸ PMBC over a commercial anti-Ig affinity column (US Biotek Laboratories, Seattle, Wash.) and 2×10⁵ responder cells are subsequently cultured with 2×10⁵ autologous ¹³⁷Cs-irradiated (2500 rads) non-T stimulator cells from a single donor. The medium employed consists of RPMI-1640 supplemented with 20 mM HEPES (Invitrogen), 5×10⁻⁵ M 2-mercaptoethanol (BDH, Montreal, QC), 4 mM L-glutamine (Invitrogen), 100 U/ml penicillin (Invitrogen), and 100 μg/ml streptomycin sulfate, with the addition of 10% fresh human serum autologous to the responder T-cell donor for the AMLR. Varying concentrations of purified recombinant human AFP, human serum albumin, anti-human AFP monoclonal antibody clone #164 (125 μg/ml final concentration in culture) (Leinco Technologies, St. Louis, Mo.) are added at the initiation of cultures. AMLR cultures are incubated for 4 to 7 days, at 37° C. in 95% air and 5% CO₂. At the indicated intervals, DNA synthesis is assayed by a 6 hour pulse with 1 μCi of ³H-thymidine (specific activity 56 to 80 Ci/mmole; ICN Radioisotopes, Cambridge, Mass.). The cultures are harvested on a multiple sample harvester (Skatron, Sterling, Va.), and the incorporation of ³H-TdR is measured in a Packard 2500 TR liquid scintillation counter. Results are expressed as mean cpm the standard error of the mean of triplicate or quadruplicate cultures.

The immunosuppressive activity of a candidate AFP polypeptide or fragment within the scope of the present invention can be assessed by its ability to suppress human autologous mixed lymphocyte reactions (AMLR). Generally, the candidate AFP polypeptide, fragment, or derivative is tested for its ability to inhibit the proliferative response of autoreactive lymphocytes stimulated by autologous non-T-cells, by measuring lymphocyte autoproliferation throughout a time course of 4 to 7 days. Suppression of AMLR in a dose-dependent manner is demonstrated by results from dose-response studies performed at the peak of T-cell autoproliferation where an AFP polypeptide or fragment is added at the initiation of cultures. Furthermore, parallel viability studies can be used to establish that the inhibitory activity of an AFP polypeptide or fragment on human autoreactive T-cells is not due to non-specific cytotoxic effects.

A third assay of AFP polypeptide or fragment activity can be preformed using a myelin oligodendrocyte glycoprotein (MOG) mouse model of experimental autoimmune encephalomyelitis (EAE). In the assay, mice are immunized with MOG, which leads to the development of EAE in the animals. A candidate AFP polypeptide or fragment is administered to a selected group of mice on a daily basis, beginning prior to, at the same time, or subsequent to the start of the administration of MOG to the animals. The symptoms of EAE in these animals are monitored and compared to those in a control group (e.g., those receiving only saline injections) over a certain time period, e.g., 30 days. Severity of EAE in each animal is given a score between 1-5 based on defined symptoms and the average score of animals in a group indicates the disease state of the group. Biologically active AFP proteins or fragments will reduce the severity of EAE in animals receiving MOG compared to controls.

### DMARDS

Several anti-arthritic drugs are known in the field and presently used to treat patients with inflammatory arthritic diseases. Examples of DMARDs known in the art include, but are not limited to auranofin, aurothioglucose, azathioprine, chlorambucil, cyclophosphamide, cyclosporine; D-penicillamine, gold sodium thiomalate (injectable gold), hydroxychloroquine, leflunomide, methotrexate, minocycline, mycophenolate mofetil, or sulfasalazine.

Methotrexate is an example of a DMARD that can be used in one embodiment of the combination treatment method of this invention. Methotrexate, also known as Amethopterin, RHEUMATREX® (Lederle Pharmaceutical), or FOLEX® (Aventis), is an antimetabolite that competitively and reversibly inhibits dihydrofolate reductase (DHFR), an enzyme that is part of the folate synthesis metabolic pathway. The affinity of methotrexate for DHFR is about one thousand-fold that of folate for DHFR, which catalyses the conversion of dihydrofolate to the active tetrahydrofolate. Folic acid is needed for the de novo synthesis of the nucleoside thymidine, required for DNA synthesis. Methotrexate is therefore capable of inhibiting the synthesis of DNA, RNA, and thymidylates. Targeting the S-phase of the cell cycle, methotrexate has a greater negative effect on rapidly dividing cells. As a result, methotrexate has been prescribed for treating a number of medical conditions including certain cancers, severe psoriasis, and inflammatory arthritic diseases.

The chemical name for methotrexate is N-[4-[[(2,4-diamino-6-pteridinyl)methyl]methylamino]benzoyl]-L-glutamic acid, although it is commonly present in the form of a sodium salt in pharmaceutical compositions and its amount in such compositions is determined by equivalence to the free acid. Therefore, when a composition is said to contain 10 mg of methotrexate, a greater weight of a sodium salt of methotrexate may be present in the composition. Methotrexate is a generic drug that has been in use for many years and is commercially available through various suppliers. For instance, methotrexate is manufactured and marketed by both Pfizer and Wyeth.

### Pharmaceutical Compositions

The present invention also relates to a pharmaceutical composition that contains a therapeutically effective amount of an AFP (or its functional fragment) and/or a DMARD. The active ingredients, the AFP (or its biologically active fragment) and the DMARD, may be present in the same pharmaceutical composition (a single dosage form) or separate pharmaceutical compositions (separate dosage forms). The compositions can be formulated for use in a variety of drug delivery systems. One or more physiologically acceptable exicipients or carriers can also be included in the compositions for proper formulation. Suitable formulations for use in the present invention are found in Remington's Pharmaceutical Sciences, Mack Publishing Company, Philadelphia, Pa., 17th ed., 1985. For a brief review of methods for drug delivery, see, Langer, Science 249:1527-1533, 1990.

The pharmaceutical compositions are intended for parenteral, intranasal, topical, oral, or local administration, such as by a transdermal means, for prophylactic and/or therapeutic treatment. Commonly, the pharmaceutical compositions are administered parenterally (e.g., by intravenous, intramuscular, or subcutaneous injection), or by oral ingestion, or by topical application or intraarticular injection at areas affected by the inflammatory arthritic disease. Thus, the invention provides compositions for parenteral administration that comprise the AFP (or biologically active fragment thereof) and/or the DMARD dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the AFP (or its biologically active fragment) and/or the DMARD, such as in a sealed package of tablets or capsules. The composition in solid form can also be packaged in a container for a flexible quantity, such as in a squeezable tube designed for a topically applicable cream or ointment.

The compositions containing an effective amount of the AFP (or its biologically active fragment) and/or the DMARD can be administered for prophylactic and/or therapeutic treatments. In therapeutic applications, compositions are administered to a patient already suffering from an inflammatory arthritic disease in an amount sufficient to cure or at least partially arrest the symptoms of the disease and its complications. An amount adequate to accomplish this purpose is defined as a “therapeutically effective dose.” Amounts effective for this use may depend on the severity of the disease or condition and the weight and general state of the patient, but generally range from about 0.5 mg to about 100 mg of AFP (or its biologically active fragment) and from about 0.1 mg to about 2000 mg of DMARD per dose per patient.

The patient may also receive an AFP (or a biologically active fragment thereof) in the range of about 0.5 mg to about 100 mg per dose one or more times per week (e.g., 2, 3, 4, 5, 6, or 7 times or more per week), preferably about 5 mg to about 75 mg per dose per week, more preferably about 10 to about 50 mg per dose per week, and even more preferably about 20 mg to about 40 mg per dose per week.

During the above treatment, a patient may also receive a DMARD in the range of about 0.1 to 3,000 mg per dose one or more times per week (e.g., 2, 3, 4, 5, 6, or 7 or more times per week), 0.1 to 2,500 mg per dose per week, 0.1 to 2,000 mg per dose per week, 0.1 to 1,000 mg per dose per week, 0.1 to 750 mg per dose per week, 0.1 to 500 mg per dose per week, 0.1 to 250 mg per dose per week, or 0.1 to 100 mg per dose per week.

The co-administration of AFP (or its biologically active fragment) and a DMARD according to the method of this invention refers to the use of the two active ingredients in the same general time period or using the same general administration method. It is not always necessary, however, to administer both at the same time or in the same way. For instance, if an AFP and a DMARD are administered to a patient suffering from an inflammatory arthritic disease in two separate pharmaceutical compositions, the two compositions treated need not be delivered to the patient during the same time period or even during two partially overlapping time periods. In some cases, the administration of the second agent (i.e., an AFP polypeptide) may begin shortly after the completion of the administration period for the first agent (e.g., methotrexate), or vice versa. Such time gap between the two administration periods may vary from one day to one week, to one month, or more. In some cases, one therapeutic modality (e.g., an AFP polypeptide) may be administered first with the second (e.g., methotrexate) in a time period, and subsequently administered without the second in a following period. A typical schedule for this type may require a higher dosage of the first therapeutic modality in the first, co-administrative period, and a lower dosage in the second period.

Single or multiple administrations of the compositions comprising an effective amount of an AFP and/or a DMARD (e.g., methotrexate) can be carried out with dose levels and pattern being selected by the treating physician. The dose and administration schedule can be determined and adjusted based on the severity of the inflammatory arthritic disease in a patient, which may be monitored throughout the course of treatment according to the methods commonly practiced by clinicians or those described herein.

In addition compositions may further comprise more than one DMARD, which can be administered according to the same methods described above.

### Kits

The invention also provides kits for treating the symptoms of inflammatory arthritic disease according to the combination treatment method of the present invention. The kits typically include a pharmaceutical composition containing an AFP polypeptide or a biologically active AFP fragment as well as a pharmaceutical composition containing a DMARD, each in a therapeutically effective amount for treating an inflammatory arthritic disease. In one example, effective amounts of the AFP or its biologically active fragment and the DMARD can be present in a single pharmaceutical composition. Optionally, the pharmaceutical composition(s) may contain one or more pharmaceutically acceptable exicipients.

Preferably, the kits include multiple packages of the single-dose pharmaceutical composition(s) containing an effective amount of an AFP (or a biologically active fragment thereof) and/or a DMARD (e.g., methotrexate). Optionally, instruments or devices necessary for administering the pharmaceutical composition(s) may be included in the kits. For instance, a kit of this invention may provide one or more prefilled syringes containing an effective amount of an AFP polypeptide (or a biologically active fragment thereof) and one or more prefilled syringes or tablets containing an effective amount of a DMARD (e.g., methotrexate). Furthermore, the kits may also include additional components such as instructions or administration schedules for a patient suffering from an inflammatory arthritic disease to use the pharmaceutical composition(s) containing an AFP polypeptide (or a biologically active AFP fragment) and/or a DMARD. The kit may also include more than one DMARD.

It will be apparent to those skilled in the art that various modifications and variations can be made in the compositions, methods, and kits of the present invention without departing from the spirit or scope of the invention. Thus, it is intended that the present invention cover the modifications and variations of this invention provided they come within the scope of the appended claims and their equivalents.

### EXAMPLES

The following examples are provided by way of illustration only and not by way of limitation. Those of skill in the art will readily recognize a variety of non-critical parameters that could be changed or modified to yield essentially the same or similar results.

### Example 1

### Functional Test of a Recombinant AFP Using MOG-EAE Mouse Model

Efficacy experiments of a recombinant version of human AFP were performed in a mouse model in which experimental autoimmune encephalomyelitis (EAE) is induced by immunization of mouse with myelin oligodendrocyte glycoprotein (MOG).

Purpose of Study: The purpose of these studies was test compounds intended as therapeutics to treat an autoimmune disease directly associated with the major histocompatibility complex (MHC) class II molecule HLA-DR2.

EAE Model Description and Features: Experimental Allergic Encephalomyelitis (EAE) is a demyelinating disease of the central nervous system. It serves as the animal model for Multiple Sclerosis (MS) (Goverman, Lab. Anim. Sci. 46:482, 1996; Paterson, Clin. Immunol. Rev. 1:581, 1981). EAE can assume an acute, chronic, or relapsing-remitting disease course that is dependent upon the method of induction and type of animal used. Disease induction results in escalating degrees of ascending animal paralysis. The resulting paralysis is debilitating, but not painful, and most animals will show some degree of recovery even from advanced stages of EAE. Paralysis usually begins with a weakened tail, gradually followed by hind limb weakness progressing to paralysis, and less frequently front limb paralysis. EAE disease progression can be monitored with a scoring system that starts with the normal condition and ends when the mice become moribund. Since the severity of the disease varies from animal to animal there is no way to reliably predict whether an animal will recover. As a result, close monitoring is needed in this animal model.

EAE can be induced with components of the central nervous system (Levine and Sowinski, J. Immunol. 110:139, 1973; Fritz et al., J. Immunol. 130:1024, 1983) or peptides (Tuohy et al., J. Immunol. 140:1868, 1988; McFarlin et al., Science 179:478, 1973; and Linington et al., Eur. J. Immunol. 23:1364, 1993) and also via T-cell transfer from one animal to another animal (Yamamura et al., J. Neurol. Sci. 76:269, 1986). Complete Freund's Adjuvant (CFA) must be used with the proteins or peptides to effectively trigger the autoimmune response. CFA is often used in combination with pertussis toxin (Lee, Proc. Soc. Exp. Biol. Med. 89:263, 1955; Kamradt et al., J. Immunol. 147:3296, 1991) to increase the efficiency of immunization. It is not possible to administer analgesics to lessen any pain that may be associated with the CFA injections, as most analgesics affect the immune response that is an essential component of the model (Billiau, J. Leukoc. Biol. 70:849, 2001; Naiki et al., Int. J. Immunopharmacol. 13:235, 1991).

### Experimental Design and Methods

Induction of experimental MS-like disease syndrome: Fifty female mice (C57BL6) between 6 and 8 weeks of age, were immunized subcutaneously on day 0 (left paralumbar region) and day 7 (right paralumbar region) with an emulsion (125 μg per mouse) of myelin oligodendrocyte glycoprotein (mMOG-35-55 peptide) in CFA containing heat-killed Mycobacterium tuberculosis H37RA. In addition, mice were given pertussis toxin (Ptx) intraperitonealy, on days 0 and 2 post-immunization.

Disease monitoring: The initial signs of disease (weakened tail or paralysis) were observed beginning ˜10 days after first immunization. Actively immunized mice were assessed daily through day 30 for clinical signs of EAE according to an established scale:
1 Tail weakness
2 Hind limb paralysis sufficient to impair righting or 1 limb paralysis
3 Parapalegic
4 Quadrapeligic
5 Moribund or dead

The 50 mice were randomized into 5 groups of 10 mice each. One group of 10 animals received a saline injection to serve as an untreated EAE disease control. Four compounds were evaluated in the remaining 4 groups.

Mice were injected with 100 μl of test recombinant hAFP (rhAFP) or control material IP daily. These compounds are: 1-500 μg rhAPF or 1-500 μg human serum albumin (control). Furthermore, depleting antibodies to specific cell subsets (e.g., CD4⁺ cells) are employed as additional control(s) in some studies.

Mice were used in this study to assess the effect of rhAFP on disease progression in an experimental model of MS (EAE). Without treatment it was expected that many of the animals would develop signs and symptoms of EAE, namely progressive encephalopathy and paralysis.

In addition to daily monitoring of the animals for disease progression over a 30-day time course, animals were sacrificed at the end of the study and central nervous system tissues (brain and spinal cord) were harvested for immunohistochemical analysis of infiltrating, disease causing cells (i.e., CD4⁺ T cells).

Additionally, six to ten-day short-term studies were employed to assess the effect(s) of rhAFP administration on the induction phase of disease. In these shorter studies, draining lymph node cells were harvested for FACS analysis of immunologic cell subsets including but not limited to: T cells, CD4⁺ cells, regulatory T cells, and their activation markers. A fraction of harvested cells from each treatment group were assessed for in vitro proliferative response to a panel of stimuli to assess antigen-specific recall response to the immunizing antigen, MOG35-55 and antigen-nonspecific responses to a panel of mitogens (Conconavalin A, PHA, LPS). Supernatants from cultures set-up in the same fashion are analyzed for cytokine (IL-2, IL-4, IFNγ, etc.).

### Example 2

### Synergistic Effect of an AFP and a DMARD in Inhibiting TNF-α Production in Mouse Splenocytes

The effect of an AFP and a DMARD (e.g., methotrexate) for inhibiting inflammation is tested in an in vitro assay measuring mouse splenocyte TNF-α production.

Assay Description and Features:

Splenocytes collected from 7-12 week old female C57BL/C mice (Charles River Labs, Wilmington, Mass.) were cultured in 96 well tissue culture plates at a density of 5×10⁶ cells per well in RPMI media (Invitrogen) supplemented with 5% fetal bovine serum and antibiotics (penicillin/streptomycin; Sigma Aldrich, St. Louis, Mo.) in a final volume of about 200 μL per well. In addition to the media, cells received phytohemagluttinin-L (PHA; Sigma) at a final concentration of 5 mg/ml. Cells were plated with media alone were also included as a negative control. After plating, cells (which are not adherent but settle to the bottom of the wells) were cultured for 24 hours in a 37° C. incubator with 5% CO₂. After about 24 hours, about 90% of the supernatant is aspirated (by manual pipetting) from the wells, leaving the cells behind. The supernatant is frozen at −20° C. for analysis later by ELISA (Murine IFN-γ ELISA kit; R&D Systems, Minneapolis, Minn.). The readout of IFN-γ is in the range of picograms/ml. The concentration of AFP is from 1 to 100 μg/mL and the concentration of methotrexate is from 1 to 20 μM.

Data Analysis:

The data for each AFP and methotrexate combination were analyzed and classified into one of three categories: additive, synergistic, or antagonistic, as described below.

Additive: The additive effect is the amount of inhibition expected when two drugs are combined together. Calculating this reference level is more complicated than simply adding the effects of the drugs when used individually. In fact, there are multiple methods for defining additivity. The most common definition is Loewe additivity (Fitzgerald et al., Nature Chem. Biol. 9:458-466, 2006), which assumes that both drugs act via a similar mechanism. For example, combining two drugs that are actually the same drug would result in a combination response that is Loewe additive. Loewe additivity is experimentally challenging as complete dose-response curves of the individual inhibitors are required for the calculation. A more tractable definition of the additive level is the Highest Single Agent (also called Gaddum's non-interaction) which is simply the maximum of the inhibition observed for each of the drugs when used individually at the concentrations used in the combination. The third major definition of additivity is Bliss Independence (Fitzgerald et al., supra), which calculates the additive level as the multiplication of the effects of the two drugs when used independently. Thus, Bliss Independence assumes independent but competing mechanisms. None of the three definitions perfectly describe the underlying biological mechanism of drugs but are useful phenomenological definitions for assessing the effectiveness of a drug combination. A combination exhibiting an additive effect may be advantageous in the clinic because the total dose of each agent can be reduced to achieve the desired potency (decreasing the likelihood of potentially harmful toxicity).

Synergistic: A mutually reinforcing drug interaction such that the joint effect of two drugs administered simultaneously is greater than expected from additive models using their individual effects. A combination exhibiting an additive effect may be advantageous in the clinic because the total dose of each agent can be reduced to achieve the desired potency (decreasing the likelihood of potentially harmful toxicity).

Antagonistic: The opposite of synergy, the combination exhibits less effect than expected based on the individual drug potencies.

Data collected are classified as additive, syngergistic, or antagonistic based on the criteria that a difference between the observed inhibition data and the additive definition of less than or equal to 5 percentage points was considered additive, a difference of greater than 5 percentage points was considered synergistic, and a difference value less than −5 percentage points was considered antagonistic. The data can be analyzed as described above using an additive definition based on the Highest Single Agent definition or the Bliss Independence definition (see, Fitzgerald et al., supra).

Results:

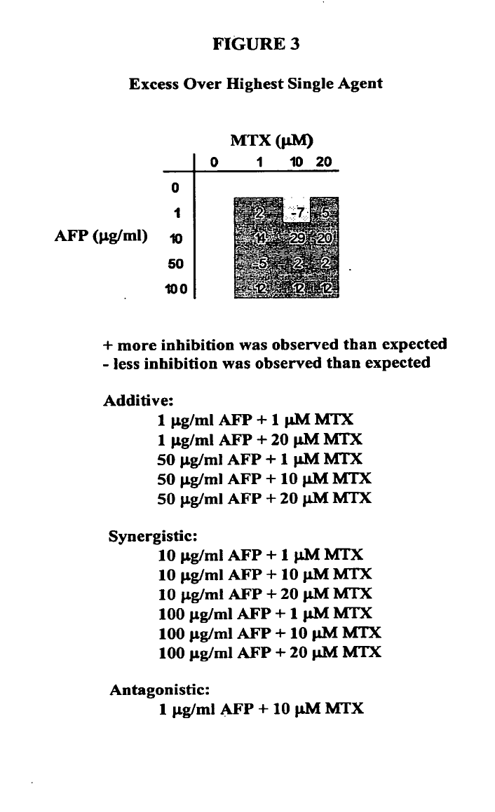

When the AFP and methotrexate data were analyzed in comparison to the Highest Single Agent definition six different dosage combinations of AFP and methotrexate (MTX) were found to be synergistic for their effect in decreasing TNF-α production in splenocytes (see, FIG. 3): 10 μg/mL AFP+1 μM MTX, 10 μg/mL AFP+10 μM MTX, 10 μg/mL AFP+20 μM MTX, 100 μg/mL AFP+1 μM MTX, 100 μg/mL AFP+10 μM MTX, and 100 μg/mL AFP+20 μM MTX.

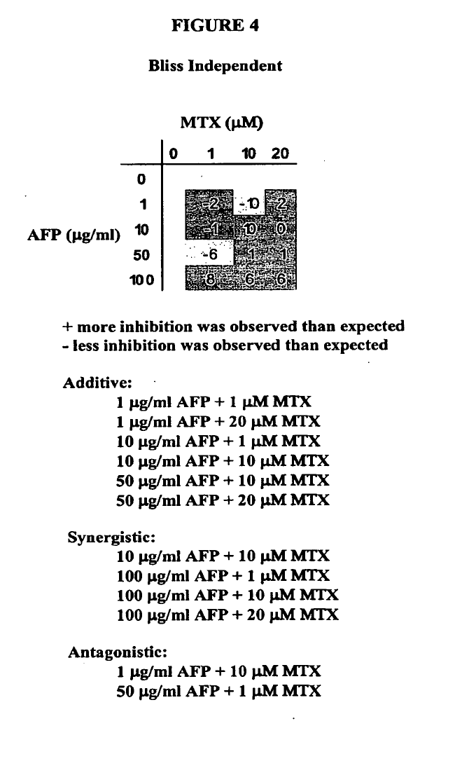

When the AFP and methotrexate data were analyzed in comparison to the Bliss Independent definition four different dosage combinations of AFP and methotrexate were found to be synergistic for their effect in decreasing TNF-α production in splenocytes (see, FIG. 4): 10 μg/mL AFP+10 μM MTX, 100 μg/mL AFP+1 μM MTX, 100 μg/mL AFP+10 μM MTX, and 100 μg/mL AFP+20 μM MTX.

### Example 3

### Synergistic Effect of an AFP and a DMARD in Treating Collagen-Induced Arthritis in Mouse

The effect of recombinant human AFP (rhAFP) and a DMARD (e.g., methotrexate) for treating rheumatoid arthritis is tested in a study utilizing a mouse model for collagen-induced arthritis (CIA).

CIA Model Description and Features: Collagen-induced arthritis (CIA) in the mouse was first described by Courtnay et al. (Nature 283:666-668, 1980). Susceptible strains of mice immunized with heterologous collagen type II (CII) develop an autoimmune polyarthritis that shares clinical, histological, and immunological features with human rheumatoid arthritis. The immune response to CII is characterized by both the stimulation of collagen specific T-cells and production of high titers of antibody for both the immunogen (heterologous CII) and the auto-antigen (mouse CII). Arthritic joints show all the hallmarks of the human disease such as cell infiltration, pannus formation, and cartilage destruction, followed by healing by fibrosis and ankylosis of involved joints.

Disease susceptibility is strongly linked to the class II molecules of MHC, specifically to the I-A^{q} and I-A^{r} molecules (Wooley et al., J. Exp. Med. 154:688-700, 1981). H-2^{q} mice such as DBA/1 and B10Q are susceptible to CIA when immunized with bovine, chicken and human CII. The incidence of arthritis is >80% and male mice have higher incidence than females.

The most widely used system for assessment of arthritis development is a simple visual scoring of 0 to 4. Any or all four paws can be affected. The inflammation of the paws is characterized by swelling and erythema. Animals are scored 2-3 times a week following this scoring system:

| Severity | |
|---|---|
| score | Gross pathology |
| 0 | No evidence of erythema or swelling |
| 1 | Erythema and mild swelling confined to the mid foot (tarsals) |
| | or ankle joints |
| 2 | Erythema and mild swelling extending from the ankle to the |
| | mid-foot |
| 3 | Erythema and moderate swelling extending from the ankle to |
| | the metatarsals joints |
| 4 | Erythema and severe swelling encompass the ankle, foot, and |
| | digits |

### Experimental Protocol

At 6-8 weeks of age DBA/1 mice are immunized subcutaneously in the base of the tail with 200 μg of heterologous CII emulsified (1:1) in Complete Freund's Adjuvant containing 4 mg/ml of Mycobacterium tuberculosis (Garcia et al., J. Autoimmunity 13:315-324, 1999). Three weeks later, the mice receive a booster with 50 μg CII emulsified in Incomplete Freund's Adjuvant. Clinical disease activity is assessed 3 times a week between days 21 and 60 after immunization following the scoring system described above. Disease onset is expected one week after the booster.

### Treatment Regimen

Groups of 10 mice are immunized at days 0 and 21 with type II collagen. In addition, the mice of group 1 receive a placebo; group 2 receives AFP at 10 mg/week; group 3 receives AFP at 20 mg/week; group 4 receives methotrexate at 10 mg/week; group 5 receives methotrexate at 20 mg/week; group 6 receives both AFP and methotrexate, each at 10 mg/week; and group 7 receives both AFP and methotrexate, each at 20 mg/week. The administration is by daily intraperitoneal injections (Neurath et al., Clin. Exp. Immunol. 115:42-55, 1999; and Lange et al., Annals of Rheumatic Diseases 64:599-605, 2005) from day 0 until the end of experiment at day 60. All groups are scored for disease symptoms according to the scale as stated above twice a week for the duration of the study.

### End Point of Study

At day 60, all mice are euthanized, and various organs and blood (e.g., spleen, knees, hind and fore paws) are harvested for immuno-histochemistry and immunological analysis.

Assessment of Immune Response in CIA

At several time points serum and splenocytes are collected from a separate cohort of mice for assessment of the T- and B-cell response against CII in the presence or absence of AFP and/or methotrexate. A suspension of splenocytes is cultured ex vivo in the presence of increasing amounts of CII in a recall response. The supernatant of the cultures is analyzed for cytokine production. To further characterize the effect of AFP and/or methotrexate on T-cell activation and differentiation, splenocytes are stained with antibodies against several markers and analyzed by fluorescence-activated cell sorting (FACS). Antibodies against CII and their isotypes are measured in the serum.

All patents, patent applications, and other publications cited in this application, including published amino acid or polynucleotide sequences, are incorporated by reference in the entirety for all purposes.

What is claimed is:

1. A method of treating a patient with an inflammatory arthritic disease, comprising administering to said patient a therapeutically effective amount of an alpha-fetoprotein (AFP) or a biologically active fragment thereof and a therapeutically effective amount of a disease modifying anti-rheumatic drug (DMARD).

2. The method of claim 1, wherein said AFP or biologically active fragment thereof is human recombinant AFP.

3. The method of claim 1, wherein said AFP or biologically active fragment thereof is non-glycosylated.

4. The method of claim 1, wherein said DMARD is auranofin, aurothioglucose, azathioprine, chlorambucil, cyclophosphamide, cyclosporine, D-penicillamine, gold sodium thiomalate (injectable gold), hydroxychloroquine, leflunomide, methotrexate, minocycline, mycophenolate mofetil, or sulfasalazine.

5. The method of claim 1, wherein two or more DMARDs are administered in a therapeutically effective amount.

6. The method of claim 1, wherein said AFP or biologically active fragment thereof, and said DMARD is administered intravenously, intramuscularly, orally, by inhalation, parenterally, intraperitoneally, intraarterially, transdermally, sublingually, nasally, through use of suppositories, transbucally, liposomally, adiposally, intraocularly, subcutaneously, intrathecally, topically, or through local administration.

7. The method of claim 1, wherein said AFP or biologically active fragment thereof, or said DMARD is administered daily, weekly, biweekly, or monthly.

8. The method of claim 1, wherein said AFP or biologically active fragment thereof, and said DMARD are administered coextensively or separately.

9. The method of claim 1, wherein said AFP or biologically active fragment thereof, and said DMARD are administered in separate dosage forms.

10. The method of claim 1, wherein said AFP or biologically active fragment thereof, and said DMARD are administered in a single dosage form.

11. The method of claim 1, wherein said AFP or biologically active fragment thereof, and said DMARD are administered via two separate routes of administration.

12. The method of claim 1, wherein said inflammatory arthritic disease is osteoarthritis, rheumatoid arthritis, juvenile rheumatoid arthritis, Crohn's disease, fibromyalgia, systemic lupus erthrematosis, ankylosing spondylitis, gout, polymyalgia rheumatica, or psoriatic arthritis.

13. A composition comprising an alpha-fetoprotein (AFP) or a biologically active fragment thereof and a disease modifying anti-rheumatic drug (DMARD), each in an amount that is therapeutically effective to treat, prevent, or reduce the symptoms or progression of an inflammatory arthritic disease in a patient in need thereof.

14. The composition of claim 13, wherein said DMARD is auranofin, aurothioglucose, azathioprine, chlorambucil, cyclophosphamide, cyclosporine, D-penicillamine, gold sodium thiomalate (injectable gold), hydroxychloroquine, leflunomide, methotrexate, minocycline, mycophenolate mofetil, or sulfasalazine.

15. The composition of claim 13, wherein said composition comprises two or more DMARDs.

16. The composition of claim 13, wherein said composition is formulated for intravenous, intramuscular, oral, parenteral, intraperitoneal, intraarterial, transdermal, sublingual, nasal, transbuccal, liposomal, adiposal, intraocular, subcutaneous, intrathecal, topical, or through suppository, inhalation, or local administration.

17. The composition of claim 13, wherein said AFP or a biologically active fragment thereof is human recombinant AFP.

18. The composition of claim 13, wherein said AFP or a biologically active fragment thereof is non-glycosylated.

19. The composition of claim 13, wherein said inflammatory arthritic disease is osteoarthritis, rheumatoid arthritis, juvenile rheumatoid arthritis, Crolm's disease, fibromyalgia, systemic lupus erthrematosis, ankylosing spondylitis, gout, polymyalgia rheumatica, or psoriatic arthritis.

20. A kit comprising an alpha-fetoprotein (AFP) or a biologically active fragment thereof and a disease modifying anti-rheumatic drug (DMARD), each in a therapeutically effective amount to treat, prevent, or reduce the symptoms or progression of an inflammatory arthritic disease in a patient in need thereof, and instructions for administration to said patient.

21-27. (canceled)


SEQUENCE LISTING (12 sequences)

SEQ ID NO: 1 - protein, 591 aa, Homo sapien
RTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 2 - DNA, 2032 bp, Homo sapien
tccatattgtgcttccaccactgccaataacaaaataactagcaaccatgaagtgggtggaatcaatttttttaattttcctactaaattttactgaatccagaacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttatgtatcagacatgaaatgactccagtaaaccctggtgttggccagtgctgcacttcttcatatgccaacaggaggccatgcttcagcagcttggtggtggatgaaacatatgtccctcctgcattctctgatgacaagttcattttccataaggatctgtgccaagctcagggtgtagcgctgcaaacgatgaagcaagagtttctcattaaccttgtgaagcaaaagccacaaataacagaggaacaacttgaggctgtcattgcagatttctcaggcctgttggagaaatgctgccaaggccaggaacaggaagtctgctttgctgaagagggacaaaaactgatttcaaaaactcgtgctgctttgggagtttaaattacttcaggggaagagaagacaaaacgagtctttcattcggtgtgaacttttctctttaattttaactgatttaacactttttgtgaattaatgaaatgataaagacttttatgtgagatttccttatcacagaaataaaatatctccaaatg

SEQ ID NO: 3 - protein, 8 aa, Homo sapien
EMTPVNPG

SEQ ID NO: 4 - protein, 34 aa, Homo sapien
LSEDKLLACGEGAADIIIGHLCIRHEMTPVNPGV

SEQ ID NO: 5 - protein, 197 aa, Homo sapien
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRES

SEQ ID NO: 6 - protein, 192 aa, Homo sapien
SLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQES

SEQ ID NO: 7 - protein, 201 aa, Homo sapien
QALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 8 - protein, 389 aa, Homo sapien
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQES

SEQ ID NO: 9 - protein, 393 aa, Homo sapien
SLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 10 - protein, 331 aa, Homo sapien
QDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 11 - protein, 609 aa, Homo sapien
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 12 - protein, 591 aa, Homo sapien
  MISC_FEATURE (233)..(233): Xaa = any amino acid except Asparagine
RTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVXFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV